(19) United States
(12) Patent Application Publication
(10) Pub. No.: US 2024/0058520 A1
(43) Pub. Date: Feb. 22, 2024

(54) WHEELCHAIR SECURABLE URINE COLLECTION SYSTEMS AND RELATED METHODS

(71) Applicant: PureWick Corporation, Covington, GA (US)

(72) Inventors: Zhihui Yin, Lilburn, GA (US); Matthew Jordan Rothberg, Atlanta, GA (US); Clayton Lewis Compton, Conyers, GA (US)

(21) Appl. No.: 18/260,394

(22) PCT Filed: Jan. 6, 2022
(86) PCT No.: PCT/US2022/011419

§ 371 Date: Jul. 5, 2023

Related U.S. Application Data
(60) Provisional application No. 63/134,632, filed on Jan. 7, 2021.

(51) Int. Cl. A61M 1/00; A61F 5/453; A61F 5/455; A61F 5/44; A61F 5/441

(52) U.S. Cl. CPC A61M 1/62; A61F 5/453; A61F 5/455; A61F 5/4405; A61F 5/441; A61M 2202/04; A61M 2209/084; A61M 2205/128; A61M 2205/125; A61M 2205/18; A61M 2205/3327; A61M 2205/3389; A61M 2205/3561; A61M 2205/3592; A61M 2205/50; A61M 2205/8206

(57) ABSTRACT

Examples relate to portable urine collection systems and methods of use and assembly. The portable urine collection system (10) may include a urine collection device (12) configured to be positioned at least proximate to a urethra of a user, a conduit (17) in fluid communication with the urine collection device, a urine collection container (14) having an interior region, a pump (16) secured to the urine collection container and configured to promote urine from the urine collection device through the conduit into the urine collection container, and a sensor secured to the container and configured to detect a property relating at least to a volume of the urine in the urine collection container.

### CROSS-REFERENCE TO RELATED APPLICATIONS

This application claims priority to U.S. Provisional Patent Application No. 63/134,632 filed on Jan. 7, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to fluid collection devices and methods of using fluid collection devices. In an embodiment, a portable urine collection system is disclosed. The portable urine collection system may include a urine collection device configured to be positioned at least proximate to a urethra of a user, a conduit in fluid communication with the urine collection device, a urine collection container having an interior region, a pump secured to the urine collection container and configured to promote urine from the urine collection device through the conduit into the urine collection container, and a sensor secured to the container and configured to detect a property relating at least to a volume of the urine in the urine collection container.

In an embodiment, a method of assembling a portable urine collection system is disclosed. The method includes positioning a urine collection container having a pump and a battery secured thereto within a pouch of a wheelchair. The method also includes positioning a urine collection device proximate to a urethra of a user. The method also includes fluidly coupling the urine collection device to the urine collection container with a conduit with the pump positioned to promote urine from the urine collection device through the conduit into the urine collection container.

In an embodiment, a portable urine collection system includes a urine collection container, a pump, a battery, and a sensor. The urine collection container is a generally rectangular urine collection container having an interior region, an upper surface, and a neck protruding from the upper surface. The pump is mounted to the upper surface of the urine collection container and is configured to promote urine from a urine collection device through a conduit into the urine collection container. The battery mounted to the upper surface of the urine collection container and operably coupled to the pump. The urine collection container, the battery, and the pump together form a generally rectangular profile that is sized and dimensioned to fit within a pouch on a wheelchair. The sensor is secured to the container and configured to detect a property relating at least to a volume of the urine in the urine collection container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a portable urine collection system, according to an embodiment.

FIG. 2A is a rear isometric view of a portable urine collection system secured to a wheelchair, according to an embodiment.

FIG. 2B is the portable urine collection system of FIG. 2A without the wheelchair, according to an embodiment.

FIG. 3 is a portable urine collection system, according to an embodiment.

FIG. 4 is a flow diagram of a method for assembling a portable urine collection system, according to an embodiment.

FIG. 5 is a block diagram of a controller in a urine collection system, according to an embodiment.

### DETAILED DESCRIPTION

Embodiments disclosed herein are related to fluid collection devices and methods of using the same. The devices and systems disclosed herein are configured to collect fluids from an individual. The fluids collected by the fluid collection devices may include at least one of urine, vaginal discharge, penile discharge, reproductive fluids, blood, sweat, or other bodily fluids. Embodiments disclosed herein are related to wheelchair mountable urine collection systems and related methods. Many users of urine collection devices are over 65 years old with limited mobility, often relying on wheelchairs as a primary mode of transportation. Many users also spend a significant amount of their day in a seated or supine position. Users and caregivers, then, are benefited from a urine collection system that may be both discrete and mobile, allowing users to use the urine collection system to collect urine both at home and on the go.

In at least one, some, or all of the embodiments described herein, configurations of urine collection systems are compact and result in the technical effect of allowing the urine collection systems to be selectively and quickly secured or mounted to a wheelchair. Embodiments of the urine collection systems described herein are mobile and discreet, resulting in the technical effect of allowing a user to participate in social activities without alerting others to the incontinence of the users. In many embodiments, the urine collection system also includes an alert system configured to communicate with an electronic device to alert a user or caregiver to empty the urine collection container when the urine level approaches a predetermined level, to change or recharge a battery, and/or adjust a vacuum or suction level of the pump in the urine collection system.

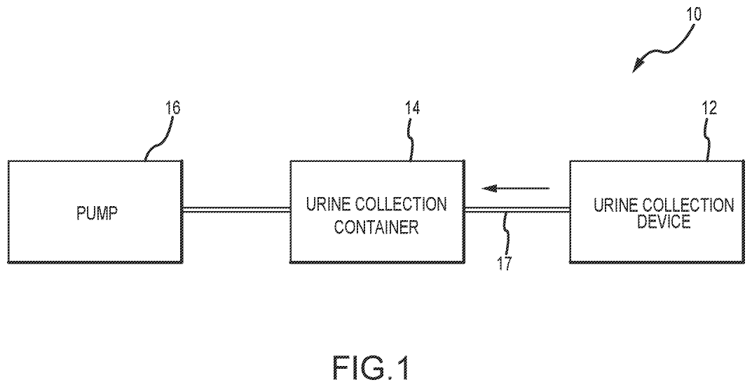

FIG. 1 is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection assemblies disclosed herein), a urine collection container 14, and a portable pump 16. The fluid collection device 10, the urine collection container 14, and the pump 16 may be fluidly coupled to each other via one or more conduits 17. For example, fluid collection device 10 may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 10 may be removed from the fluid collection device 10 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle or cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, at least a partial vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

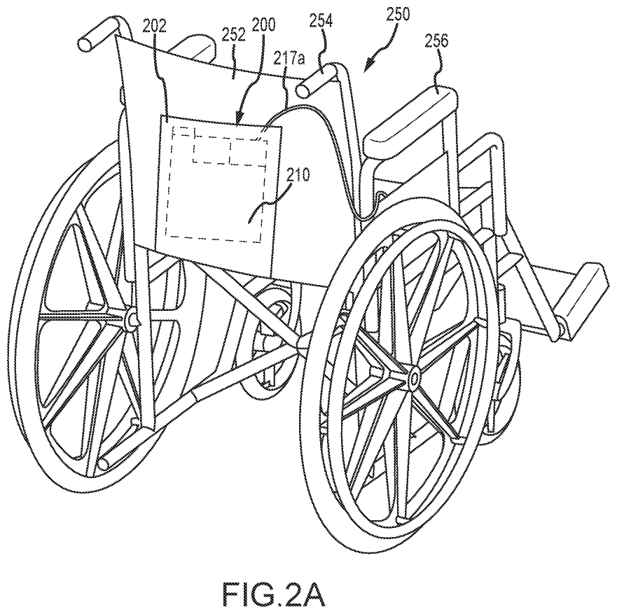

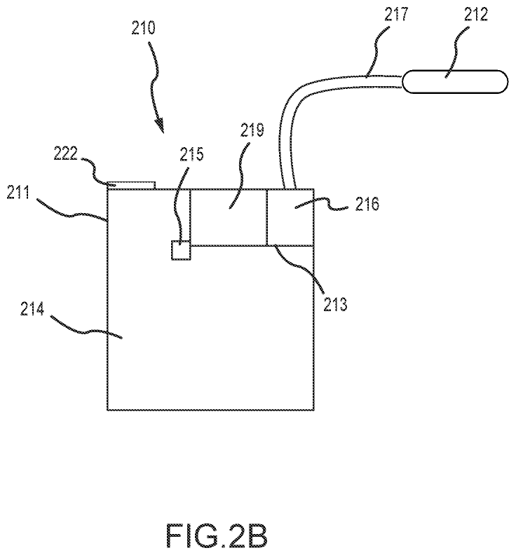

At least one, some, or all of the embodiments of urine collection systems described herein include configurations resulting in the technical effect of allowing the urine collection systems to be selectively securable or mountable to a wheelchair. Turning to FIG. 2A, a urine collection system 200 is shown secured or mounted to a wheelchair 250. The wheelchair 250 may include any of a number of different conventional wheelchairs, and may include a back 252, two handles 254, and two arms 256. In some embodiments, the wheelchair 250 may include a pouch 202 on the back 252 of the wheelchair 250 that may support a storage system 210 of the urine collection system 200. The pouch 202 may be fixedly or removably secured to the back 252 of the wheelchair 250. In some embodiments (not shown), the urine collection system 200 may include a container support in addition or alternative to the pouch 202 that is configured to detachably secure, mount, or hang from the wheelchair 250 and support a storage system 210 of the urine collection system 200. For example, the container support may include a pack or bag having one or more straps configured to hang from or secure to one or more handles 254 of the wheelchair 250.

The urine collection system 200 may include a urine collection device 212 (shown in FIG. 2B), a conduit 217, and a storage system 210. The urine collection device 212 may include a male or female urine collection device configured to be positioned at least proximate to a urethra of a user. PCT International Application No. PCT/US2019/029616 describes various embodiments of both male and female fluid collection devices, the disclosure of which are incorporated in their entirety by this reference. Moreover, the urine collection device 212 may be interchangeable in the urine collection system 200 between different types, varieties, and sizes of male or female urine collection devices. Generally, the urine collection device 212 may include a surface sized to be positioned proximate or adjacent to the urethra and configured to wick urine or other fluids away from the user. Urine or other fluids may be wicked from the surface to a reservoir in the urine collection device 212.

The urine collection system 200 also includes a conduit 217 in fluid communication with an interior region (e.g. reservoir) of the urine collection device 212 and an interior region of the urine collection container 214. The conduit 217 may be positioned between the urine collection device 212 and the urine collection container 214. In some embodiments (not shown), the urine collection system 200 also may include an additional conduit providing fluid communication between the pump 216 and the interior region of the urine collection container 214. In some embodiments, the pump 216 may be secured directly to the urine collection container 214, and the additional conduit may be absent from the urine collection system 200. The conduit 217 may include a flexible tube. In some embodiments, at least a portion of the conduit 217 is substantially opaque, thereby inhibiting viewing of the urine within the first conduit 217.

Turning to FIG. 2B, the urine collection system 200 also includes a storage system 210. The storage system 210 may include the urine collection container 214 having an interior region that stores urine received from the urine collection device 212 via the conduit 217. The urine collection container 214 may be opaque or clear according to different embodiments. In some embodiments, the urine collection container 214 includes at least one of a handle or a spout. Urine collected in the urine collection container 214 may be emptied through the spout after removing a cap or cover. The urine collection container 214 may be reusable and dishwasher safe, and may include a generally rigid material such as polycarbonate or glass.

The urine collection container 214 may be sized and dimensioned to fit within the pouch 202 on the back 252 of the wheelchair. In some embodiments, the urine collection container 214 is generally rectangular (e.g., rectangular cuboid or right rectangular prism) and includes an upper surface 213 and a neck 211 protruding from the upper surface 213. The battery 219 and the pump 216 may be attached to and/or positioned or positionable on the upper surface 213 of the urine collection container 214. When the battery 219 and the pump 216 are secured to the urine collection container 214, the urine collection container 214, the battery 219, and the pump 216 may together form a generally rectangular profile.

As the storage system 210 may be sized and dimensioned to fit within the pouch 202 on the back 252 of the wheelchair 250, the generally rectangular profile of may have a height of about 15.2 cm to about 45.7 cm, about 15.2 cm to about 22.9 cm, about 22.9 cm to about 30.5 cm, about 30.5 cm to about 38.1 cm, or about 38.1 cm to about 45.7 cm. The generally rectangular profile may have a width of about 15.2 cm to about 45.7 cm, about 15.2 cm to about 22.9 cm, about 22.9 cm to about 30.5 cm, about 30.5 cm to about 38.1 cm, or about 38.1 cm to about 45.7 cm. The urine collection container 214 may have a depth about 2.5 cm to about 15.2 cm, about 2.5 cm to about 5.1 cm, about 5.1 cm to about 7.6 cm, about 7.6 cm to about 10.2 cm, about 10.2 cm to about 12.7 cm, or about 12.7 cm to about 15.2 cm.

The storage system 210 also may include a sensor 215 secured to the urine collection container 214 and configured to detect a property relating at least to a volume of the urine in the interior region of the urine collection container 214. In some embodiments, the sensor 215 includes a negative pressure sensor, a wet sensor, a water sensor, an ultrasonic sensor, a laser sensor, or an ultraviolet (UV) sensor. In some embodiments, the sensor 215 provides feedback of the property relating to at least the volume of the urine when the urine in the container rises to the sensor 215 and contacts the sensor 215. In some embodiments, the sensor 215 may be configured to provide a continuous or periodical feedback of the property relating at least to a volume of the urine without the sensor 215 touching the urine. The sensor 215 may be secured to the urine collection container with at least a portion of the sensor 215 inside the interior region of the urine collection container 214. In some embodiments, the sensor 215 is positioned at a top of the urine collection container 214 and pointed downwards where the urine collects in the interior region of the urine collection container 214.

In embodiments having a negative pressure sensor, wet sensor, or water sensor, the sensor 215 may be positioned within the urine collection container 214 resulting in the technical effect of sensing or detecting urine in the urine collection container 214 before the urine collection container 214 is full or at volumetric capacity. For example, the sensor 215 may be positioned between or proximate to an intersection of the upper surface 213 and the neck 211 of the urine collection container 214. The urine collection container 214, then, may include the volumetric capacity of the neck 211 as a buffer between when the sensor 215 detects the urine and when the urine collection container 214 is full or at volumetric capacity. In some embodiments, the sensor 215 may be configured to detect urine at one or more predetermined thresholds. For example, the sensor 215 may detect when urine is at 25%, 50%, 75%, 90%, and/or 95% of the total volumetric capacity of the urine collection container 214. In some embodiments, such as embodiments including a negative pressure sensor or a wet sensor, the predetermined threshold is the level of the urine in the urine collection container 214 when the urine contacts the sensor 215.

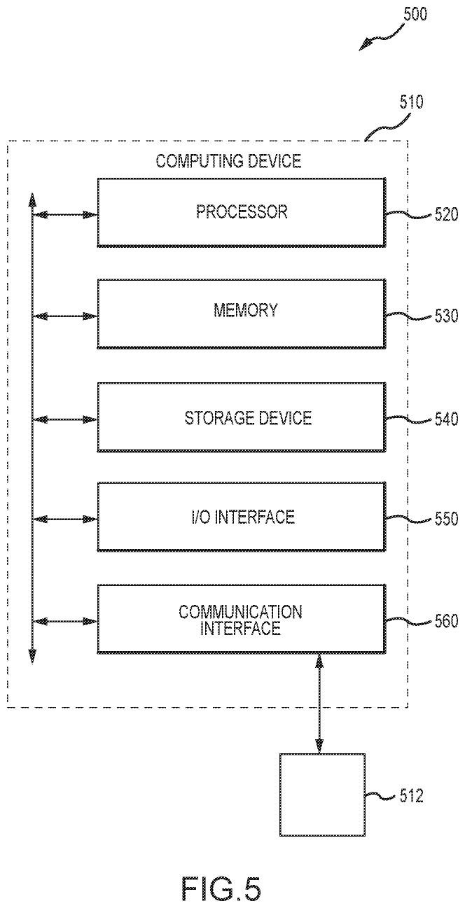

In some embodiments, the sensor 215 also includes a controller. The controller may be included with the sensor 215 or may be a separate component configured to communicate with the sensor 215. The controller may include a communication interface configured to send notifications or alerts to other electronic devices. For example, the communication interface may be configured to send notifications or alerts at a selected radio frequency, via BLUETOOH, or via WI-FI to another electronic device, such as a smartphone of the user or caregiver. Specifically, the controller may be configured to send a text message or other alert to the smartphone or other electronic device of the user or caregiver when the sensor 215 detects urine in the urine collection container 214 is at the predetermined threshold. The controller may be powered by an external or internal battery, such as a rechargeable battery. FIG. 5 provides additional details of a controller 500 that may include the controller of the urine collection system 200 or 300.

In some embodiments, the controller is configured to wirelessly transmit an alert to an electronic device of the user or a caregiver when the property relating at least to the volume of the urine detected by the sensor 215 indicates the volume of the urine in the urine collection container 215 has reached or exceeded a predetermined volume. For example, based on data from the sensor 215, the controller may wirelessly transmit an alert such as a text message to an electronic device that the urine in the urine collection container 214 has contacted the sensor 215, and emptying of the urine collection container 214 is recommended. In some embodiments, the controller may wirelessly transmit alerts and selected frequencies, such as selected time and/or volume intervals. The controller may wirelessly transmit an alert to the electronic device of the user or the caregiver when a battery powering at least one of the controller or the pump is low.

The pump 216 may include one or more of a manual vacuum pump, and electric vacuum pump, a diaphragm pump, a centrifugal pump, a displacement pump, a magnetically driven pump, a peristaltic pump, a micro pump, any combination thereof, or any pump configured to produce at least a partial vacuum. The pump 216 may provide at least a partial vacuum or suction to remove fluid from the fluid collection device 212. The pump 216 may push urine through the conduit 217. In some examples, the pump 216 may be powered by one or more batteries 219. In some examples, the pump 216 may be sized and shaped to fit within the pouch 202 with the urine collection container 214. The pump 216 may include one or more of a switch, a button, a plug, a remote, or any other device suitable to activate the pump 216, and/or the pump 216 may be configured to activate when urine is sensed in at least one of the conduit 217 or entering the urine collection container 214.

In some embodiments, the pump 216 is mounted to the urine collection container 214. For example, a peristaltic and/or micro pump may be attached to the urine collection container 214. The pump 216 may be configured to gently pull urine down from the urine collection device and/or force or push urine through the conduit 217 and into the interior region of the urine collection container 214. In some embodiments, an end of the conduit 217 may be attached to a port on the urine collection container 214 and a portion of the conduit 217 may be secured to or positioned at least partially within the pump 216. In some embodiments, the pump 216 may be in fluid communication with the interior region of the urine collection 212 and may be configured to pull at least a partial vacuum on the interior region of the urine collection container 212 effective to promote the urine from the urine collection device 212 through the conduit 217 into the urine collection container 214.

The urine collection system 200 also may include a battery 219 such as a rechargeable lithium battery. The battery 219 is configured to power at least one of the pump 216, the filter 215, and/or the controller. In some embodiments, the battery 219 includes a charge effective to provide power typically provided by an alternating current power source.

The urine collection system 200 also may include an exhaust vent 222 on the urine collection container 214 having a hydrophobic filter configured allow air from the urine collection container to exit through the exhaust vent 222 and inhibit the urine in the urine collection container from exiting through the exhaust vent 222. The exhaust vent 222 may cover the outlet or spout of the urine collection container 214. For example, the exhaust vent 222 may be positioned or positionable on the top of the neck 211 of the urine collection container 214. The filter of the exhaust vent 222 may result in the technical effect of preventing at least some odor of the urine in the urine collection container 214 from escaping the urine collection system 200. The exhaust vent 222 may allow air from the interior region of the urine collection container 214 to pass through the exhaust vent 222 when the pump 216 is activated and directing urine and/or air into the interior region of the urine collection container 214. In some embodiments, the exhaust vent 222 is removable to allow the urine collection container 214 to be emptied through an opening in the urine collection container 214 covered by the exhaust vent 222.

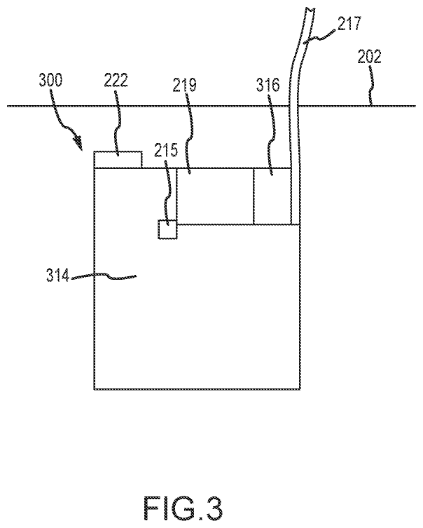

FIG. 3 is a portion of a urine collection system 300, according to an embodiment. Though not shown in FIG. 3, the urine collection system 300 may include a urine collection device. Unless otherwise noted, the urine collection system 300 may include any aspect of the urine collection system 200, such as the sensor 215, the battery 219, the exhaust vent 222, and the conduit 217. The urine collection system 300 also includes a urine collection container 314. The urine collection container 314 may include any aspect of the urine collection container 214, such as materials, shapes, and dimensions described above in relation to the urine collection container 214. The urine collection system 300 also includes a pump 316. The pump 316 may include any aspect of the pump 216 described above.

In the urine collection system 300, the pump 316 is configured to pull at least a partial vacuum on the interior region of the urine collection container 314 effective to pull at least a partial vacuum on the conduit 217 and promote urine from the urine collection device through the conduit 217 and into the interior region of the urine collection container 314. Accordingly, a portion of the pump 316 may be in fluid communication with the interior region of the urine collection container 314. In some embodiments, the pump 316 may be mounted or attached to the upper surface of the urine collection container 314. An additional conduit may fluidly couple the pump 216 with the interior region of the urine collection container 214, according to some embodiments. The conduit 217 may be secured to a port on the urine collection container 314 effective to provide fluid communication between the interior region of the urine collection device and the interior region of the urine collection container 314.

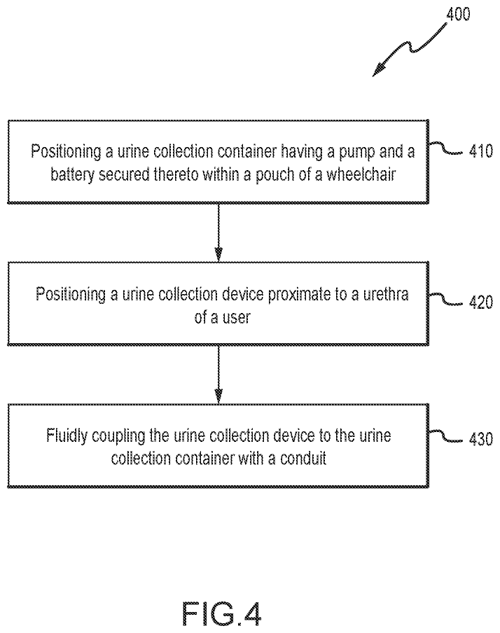

FIG. 4 is a flow diagram of a method 400 for assembling a portable urine collection system, according to an embodiment. The method 400 includes an act 410 of positioning a urine collection container having a pump and a battery secured thereto within a pouch of a wheelchair. The method 400 also includes an act 420 of positioning a urine collection device proximate to a urethra of a user. In some embodiments, the method 400 includes an act of securing the conduit to the urine collection container effective to fluidly couple the urine collection device and the urine collection container at least partially with the conduit. The method also includes an act 430 of fluidly coupling the urine collection device to the urine collection container with a conduit. The method also includes an act 430 of mounting a pump to the wheelchair. In some embodiments, the method 400 includes an act of wirelessly coupling a sensor in the container to an electronic device of a user or a caregiver. The sensor may be configured to detect a property relating at least to a volume of the urine in the urine collection container and transmit a notification to the electronic device of the user or the caregiver when the volume of the urine in the urine collection container reaches a predetermined level.

The acts of the method 400 described above are for illustrative purposes. For example, the acts of the method 400 can be performed in different orders, split into multiple acts, modified, supplemented, or combined. In an embodiment, one or more of the act of the method 400 can be omitted from the method 400. Any of the acts of the method 400 can include using any of the portable urine collection systems disclosed herein.

FIG. 5 is a schematic of a controller 500 that may be used with any of the systems and methods described herein, according to an embodiment. For example, the controller of the sensor 215 or other controller associated with the urine collection systems 200, 300 may include any aspect of the controller 500. The controller 500 may be configured to implement any of the example acts or steps disclosed herein, such as wired or wirelessly communicating with the sensor 215 or other electronic devices, and/or determining a volume of the urine in the urine collection container 214, 314.

The controller 500 includes at least one computing device 510, according to an embodiment. The at least one computing device 510 is an exemplary computing device that may be configured to perform one or more of the acts described above. The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. In particular examples, processor(s) 520 may include one or more internal caches for data such as tables pertaining to volumetric tables. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts disclosed herein or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, one or more of a history of the volume of the urine in the urine collection container, a trend of the volume of the urine in the urine collection container, a history of filter replacement, and/or a history of battery replacement or recharging may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s) to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested. For example, the at least one processor 520 may access one or more look-up tables in the memory storage medium(s) such as memory 530 or storage device 540.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI. The one or more additional computer device 512 may include a smart phone of the user, a smart phone of the caregiver, an electronic device on the wheelchair, and/or a computer device of a healthcare system.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. The computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A portable urine collection system, comprising:
a urine collection device configured to be positioned at least proximate to a urethra of a user;
a conduit in fluid communication with the urine collection device;
a urine collection container having an interior region;
a pump secured or securable to the urine collection container and configured to promote urine from the urine collection device through the conduit into the urine collection container;
an exhaust vent on the urine collection container having a hydrophobic filter configured to allow air from the urine collection container to exit through the exhaust vent and inhibit the urine in the urine collection container from exiting through the exhaust vent; and
a sensor secured or securable to the container and configured to detect a property relating at least to a volume of the urine in the urine collection container.

2. The portable urine collection system of claim 1, further comprising a battery operably coupled to the pump.

3. The portable urine collection system of claim 1, wherein the pump includes a peristaltic pump or a micro pump.

4. The portable urine collection system of claim 1, wherein the pump is secured or securable to the urine collection container between the conduit and the urine collection container.

5. (canceled)

6. The portable urine collection system of claim 1, wherein the pump is secured or securable to the urine collection container and the conduit is secured or securable to the urine collection container and configured to pull at least a partial vacuum on the urine collection container effective to pull at least a partial vacuum on the conduit and pull urine from the urine collection device through the conduit and into the interior region of the urine collection container.

7. The portable urine collection system of claim 1, wherein the urine collection container is generally rectangular and includes an upper surface and a neck protruding from the upper surface, wherein the battery and the pump are positioned or positionable on the upper surface of the urine collection container.

8. The portable urine collection system of claim 7, wherein the urine collection container, the battery, and the pump together form a generally rectangular profile.

9. The portable urine collection system of claim 8, wherein the generally rectangular profile of has a height of about 15.2 cm to about 45.7 cm and a width of about 15.2 cm to about 45.7 cm, and wherein the urine collection container has a depth about 2.5 cm to about 15.2 cm.

10. The portable urine collection system of claim 1, wherein the exhaust vent is positioned or positionable on the neck of the urine collection container.

11. The portable urine collection system of claim 7, wherein the sensor is positioned or positionable between or proximate to an intersection of the upper surface and the neck of the urine collection container.

12. The portable urine collection system of claim 1, further comprising a controller configured to communicate with the sensor and wirelessly transmit an alert to an electronic device when the property relating at least to the volume of the urine detected by the sensor indicates the volume of the urine in the urine collection container has reached or exceeded a predetermined volume.

13. The portable urine collection system of claim 1, wherein the sensor includes a wet sensor or water sensor configured to detect the urine contacting the sensor.

14. The portable urine collection system of claim 1, wherein the sensor includes an ultrasound sensor.

15. The portable urine collection system of claim 1, wherein the container and the pump are sized and dimensioned to fit within a pouch secured to a wheelchair.

16. A method of assembling a portable urine collection system, the method comprising:
positioning a urine collection container having a pump and a battery secured thereto within a pouch of a wheelchair;
positioning a urine collection device at least proximate to a urethra of a user; and
fluidly coupling the urine collection device to the urine collection container with a conduit with the pump positioned to promote urine from the urine collection device through the conduit into the urine collection container, the urine collection container including an exhaust vent having a hydrophobic filter configured to allow air from the urine collection container to exit through the exhaust vent and inhibit the urine in the urine collection container from exiting through the exhaust vent when the pump is promoting urine from the urine collection device through the conduit.

17. The method of claim 16, further comprising wirelessly coupling a sensor in the container to an electronic device of a user or a caregiver, wherein the sensor is configured to detect a property relating at least to a volume of the urine in the urine collection container and transmit a notification to the electronic device of the user or the caregiver when the volume of the urine in the urine collection container reaches a predetermined level.

18. The method of claim 16, further comprising securing the conduit to the urine collection container effective to fluidly couple the urine collection device and the urine collection container at least partially with the conduit.

19. A portable urine collection system, comprising:
a generally rectangular urine collection container having an interior region, an upper surface, and a neck protruding from the upper surface;
a pump mounted or mountable to the upper surface of the urine collection container and configured to pull and/or push urine from a urine collection device through a conduit into the urine collection container;
a battery mounted or mountable to the upper surface of the urine collection container and operably coupled to the pump, wherein the urine collection container, the battery, and the pump together form a generally rectangular profile that is sized and dimensioned to fit within a pouch on a wheelchair;
an exhaust vent on the urine collection container having a hydrophobic filter configured to allow air from the urine collection container to exit through the exhaust vent and inhibit the urine in the urine collection container from exiting through the exhaust vent; and
a sensor secured or securable to the container and configured to detect a property relating at least to a volume of the urine in the urine collection container.

20. The portable urine collection system of claim 19, wherein the pump includes a peristaltic pump or a micro pump.

21. (canceled)

22. The portable urine collection system of claim 19, wherein the exhaust vent is positioned or positionable on the neck of the urine collection container.

23. The portable urine collection system of claim 19, wherein the pump is secured or securable to the urine collection container and the conduit is secured or securable to the urine collection container and configured to pull at least a partial vacuum on the urine collection container effective to pull at least a partial vacuum on the conduit in fluid communication with the interior region of the urine collection container and pull urine from the urine collection device through the conduit and into the interior region of the urine collection container.

24. The portable urine collection system of claim 19, wherein the generally rectangular profile of has a height of about 15.2 cm to about 45.7 cm and a width of about 15.2 cm to about 45.7 cm, and wherein the urine collection container has a depth about 2.5 cm to about 15.2 cm.

25. The portable urine collection system of claim 19, wherein the sensor is positioned or positionable between or proximate to an intersection of the upper surface and the neck of the urine collection container.

26. The portable urine collection system of claim 19, further comprising a controller configured to communicate with the sensor and wirelessly transmit an alert to an electronic device when the property relating at least to the volume of the urine detected by the sensor indicates the volume of the urine in the urine collection container has reached or exceeded a predetermined volume.

27. The portable urine collection system of claim 19, wherein the sensor includes a wet sensor or water sensor configured to detect the urine contacting the sensor.

28. The portable urine collection system of claim 19, wherein the sensor includes an ultrasound sensor.
